(19) United States
(12) Patent Application Publication
(10) Pub. No.: US 2023/0100407 A1
(43) Pub. Date: Mar. 30, 2023

(54) SELF-CONTAINED ULTRAVIOLET C LIGHT GERMICIDAL LIGHT EMITTING SYSTEM AND METHOD FOR CONTINUOUS DOOR HARDWARE SANITATION

(71) Applicant: Tom CHI, South Fallsburg, NY (US)

(72) Inventor: Tom Chi, Wichita, KS (US)

(21) Appl. No.: 17/911,386

(22) PCT Filed: Mar. 14, 2021
(86) PCT No.: PCT/US2021/022261

§ 371 Date: Sep. 13, 2022

Related U.S. Application Data
(60) Provisional application No. 62/989,757, filed on Mar. 15, 2020.
(60) Provisional application No. 63/000,602, filed on Mar. 27, 2020.

(51) Int. Cl. A61L 2/10; E05B 1/00

(52) U.S. Cl. CPC A61L 2/10; E05B 1/0069

(57) ABSTRACT

A self-contained ultraviolet C light emitting system for door access includes a door for entering and exiting a premise, a door access device associated with the door, in which a user is able to come into physical contact with the door access device, a source of sanitizing ultraviolet C emitting light associated with the door access device positioned so that the door access device is sanitized and germ free; and, and a switch to electrically energize the source of the sanitizing ultraviolet light.

### BACKGROUND OF THE INVENTION

### Technical Field of the Invention

The present invention relates, generally, to a self-contained ultraviolet C germicidal light emitting system and related method for use on various surfaces for disinfecting such surfaces.

More particularly, the present invention relates generally to doorknobs, door handle levers, door bar handles and other devices that have self-contained integral disinfecting and sterilizing systems that are touched by a user's hands thereby exposing the doorknob, door lever, door bar handle, faucet lever/handle, elevator buttons and other devices and surfaces to an ultraviolet C germicidal sterilizing light source to kill or inactivate microorganisms by destroying nucleic acids, thereby disrupting their DNA or RNA and thus leaving such viruses and bacterium unable to function or reproduce.

### Description of the Prior Art

It is well established that germs from hands are easily communicated to a person's face especially when the hands touch the face several times a minute, as for example the eyes are rubbed, the nose is touched and other times when the hands consciously and unconsciously touch the face. Since the exposed face is more than any other part of the human body more susceptible to transmitting germs to the body's systems it imperative to maintain as best as possible a germ-free face and hands free of pathogens which can enter the eyes, ears, mouth, nose, etc.

Various devices and method for sanitizing surfaces and objects using ultraviolet light are generally known to the prior art and the state of the art is generally represented by the following prior art references: Visco et al., U.S. Pat. No. 4,856,140; Manson et al., U.S. Pat. No. 6,289,557; Jones, U.S. Pat. No. 7,175,807; Sassoon, U.S. Pat. No. 7,360,674; Stropkay et al., U.S. Pat. No. 7,458,742; Jones, U.S. Pat. No. 7,598,501; Zevallos, U.S. Pat. No. 7,716,789; Balzano, U.S. Pat. No. 8,353,085; Caron, U.S. Pat. No. 8,375,521; Bracey, U.S. Pat. No. 9,051,757; Brennan, U.S. Pat. No. 9,200,771; Roberts et al., U.S. Pat. No. 9,376,833; and, Strombeck et al., U.S. Pat. No. 10,422,158.

### SUMMARY OF THE INVENTION

It is, therefore, a primary object of the present invention is to provide a system for maintaining and sanitizing doorknobs, door handle levers, door bar handles, railings, train hand-holds, shopping card handles, etc. to prevent transmission of pathogens from door-knobs, door handle levers and door bar handles, etc. to a person's hand and subsequently to a person's face.

A further object of the present invention is to provide means to maintain and disinfect doorknobs, door handle levers, door bar levers by incorporating into retrofit parts for locksets or of doorknobs, levers, door-opening bars, push plates, toilet flush handles, sink levers and other knobs and buttons as well as other push-open or closed surfaces ultraviolet light to make render harmless pathogens that are transmitted by contact and spread disease.

An additional object of the present invention is to be able to be incorporated into newly manufactured doorknobs, door handle levers, door bar handles, etc., a system embedded with a source of ultraviolet C germicidal sterilizing light so that any germs, viruses, bacteria, mold and spores on the surface are rendered inactive and harmless.

Another object of the present invention is to provide constant/intermittent energy to power the ultraviolet C germicidal sterilizing light source so that the doorknobs, door handles lever and door bar handles are continuously germ free.

A still further object of the present invention is to fabricate a system from ultra-violet C germicidal sterilizing light permeable material such as quartz or ultraviolet C light stabilized ultraviolet light C permeable plastic, etc., to allow ultraviolet C germicidal sterilizing light to be in contact with the outside surface.

A further object of the present invention is to apply the ultraviolet C germicidal sterilizing light to other areas where hands are used such as toilet handles, shopping cart handles, baby strollers/carriages, sporting/recreational items etc.

Another object of the present invention is to fabricate a doorknob and/or door lever from a material that is permeable to ultraviolet C germicidal sterilizing light such as quartz and/or other type of material.

An additional object of the present invention is to adapt the system to shopping carts, baby strollers/carriages and other objects that are operated by a user's hand.

The foregoing and related objects are accomplished by the present invention, which provides a self-contained ultraviolet C light emitting system for door access, which includes a door for entering and exiting a premise and a door access device associated with the door in which a user comes into physical contact with the door access device. A source of sanitizing ultraviolet C emitting light is provided for, or otherwise associated with, the door access device positioned so that the door access device is sanitized and germ free. Further, there is an energy or power source, a movable lock barrel, preferably, an AC to DC converter, and switch means to electrically energize the source of the sanitizing ultraviolet light.

Preferably, the source of sanitizing ultraviolet C emitting light is light emitting diodes, fiber optics, glass fiber or mercury bulbs, though other ultraviolet C emitting light devices are within the scope of the present invention.

Preferably, the door access device is, or would include, a doorknob, contoured door lever, rectangular door lever, door bar handles and door push plates.

The switch means that electrically powers the door access device, preferably, includes an extended lock barrel that comes into contact and actuates a switch permitting electric current to energize the source of ultraviolet C light.

Other objects and features of the present invention will become apparent when considered in combination with the accompanying drawing figures, which illustrate certain preferred embodiment of the present invention. It should, however, be noted that the accompanying drawing figures are intended to illustrate only select preferred embodiments of the claimed invention and are not intended as a means for defining the limits and scope of the invention.

### BRIEF DESCRIPTION OF THE DRAWING FIGURES

In the drawing figures, wherein similar features are denoted with similar reference numerals throughout the several views:

FIGS. 1 and 2 are perspective views of a preferred embodiments of a doorknob containing the self-contained ultraviolet C light germicidal light emitting system of the present invention with an ultraviolet C germicidal light emitting source embedded into a doorknob;

FIGS. 3 and 4 are perspective views of a preferred embodiment of a doorknob containing the self-contained ultraviolet C light germicidal light emitting system of the present invention with an ultraviolet C germicidal light emitting source embedded into a contoured door lever handle;

FIGS. 5 and 6 are prospective views of a preferred embodiment of a doorknob containing the self-contained ultraviolet C light germicidal light emitting system of the present invention with an ultraviolet C germicidal light emitting source embedded into a rectangular door lever handle;

FIGS. 7 and 8 are perspective views of a preferred embodiment of a doorknob containing the self-contained ultraviolet C light germicidal light emitting system of the present invention with an ultraviolet C germicidal light emitting source embedded into a door bar handle;

FIGS. 9 and 10 are perspective views of a preferred embodiment of a doorknob containing the self-contained ultraviolet C light germicidal light emitting system of the present invention with an ultraviolet C germicidal light emitting source embedded into a door push plate;

FIG. 11 provides perspective views of various uses of the present invention with ultraviolet C germicidal light being emitted;

FIGS. 12A-12D provide perspective views of the present invention as incorporated into a doorknob with means for indicating whether or not the doorknob is germ-free and ready to be safely used;

FIGS. 13 and 14 are perspective views of the internal features of the present invention when not electrically energized; and

FIGS. 15 and 16 are perspective views of the internal features of the present invention when not electrically energized.

### DESCRIPTION OF THE REFERENCE NUMERALS

10 invention of an ultraviolet C germicidal light emitting doorknob
10a invention of an ultraviolet C germicidal light emitting door lever handle
10b invention of an ultraviolet C germicidal light emitting door lever handle
10c invention of an ultraviolet C germicidal light emitting door bar handle
10d invention of an ultraviolet C germicidal light emitting door push pad plate
11 access devise such as a doorknob
12 ultraviolet C germicidal light
13 door access devise such as a contoured door lever
14 cutaway view with source of ultraviolet C germicidal light
15 door access devise such as a rectangular door lever
16 source of ultraviolet C germicide light
17 door access devise such as a door bar handle
18 source of ultraviolet C germicide light
19 door access devise such as a door push pad plate
20 touching surface
22 ultraviolet C light permeable material
30 stationary door frame
32 door associated with a premise
34 striker plate
36 unengaged lock barrel
36a engaged lock barrel which provides and transmits electric power to ultraviolet C light source with wires flush and parallel to lock cylinder
38 electrical switch
40 AC-to-DC converter
42 electrical input current generally 120 volts AC.
44a to 44h system status

### DETAILED DESCRIPTION OF THE DRAWING FIGURES AND PREFERRED EMBODIMENTS

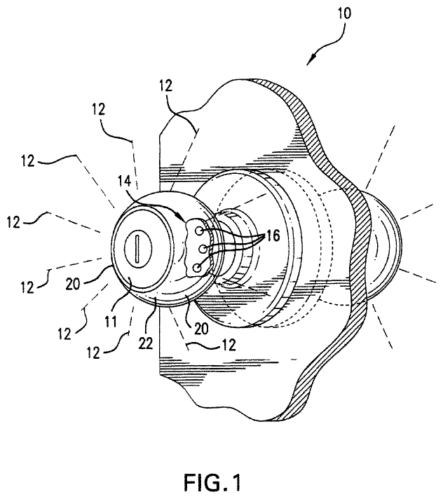

Turning now, in detail, to the accompanying drawing figures, FIG. 1 provides an overview of a preferred embodiment of the present invention 10 illustrating the principal components of a doorknob assembly 11 emitting ultraviolet C germicidal light 12. The source 16 of the ultraviolet germicidal light may be from (LED) light emitting diodes, fiber optics, glass fiber, mercury bulbs, or other ultraviolet C wavelength light sources all of which may be steady or intermittent. Cutaway 14 indicates a location of light source 16 within doorknob 11. Touching surface 20 of doorknob 11 is rendered germ-free by means of ultraviolet C germicidal light 12 being transferred to touching surface 20 through ultraviolet C light permeable material 22. Doorknob 11 may be constructed from any type of material in conjunction with ultraviolet C light permeable material 22.

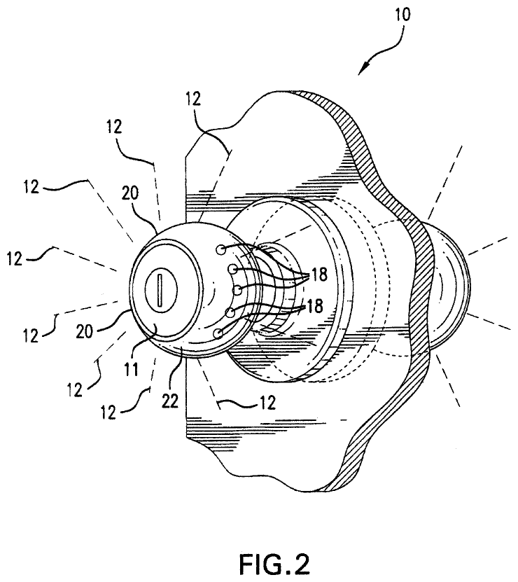

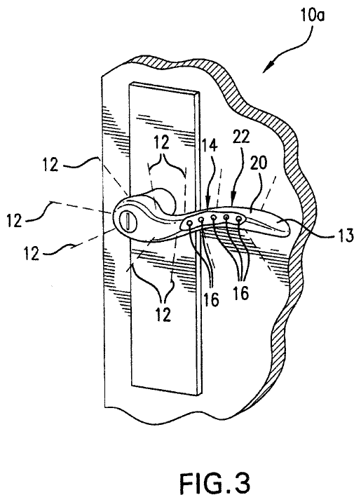

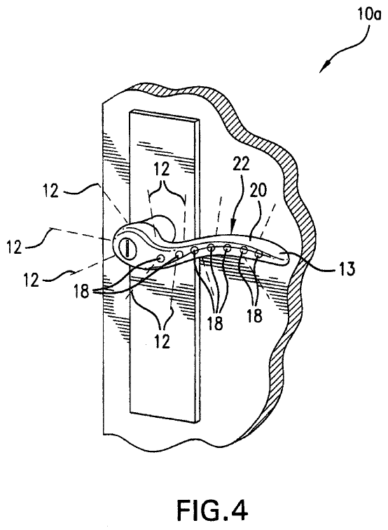

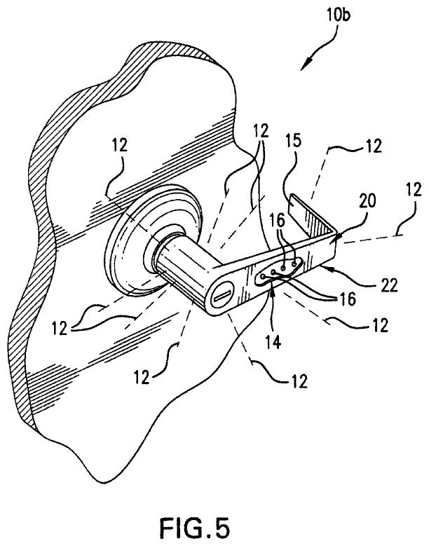

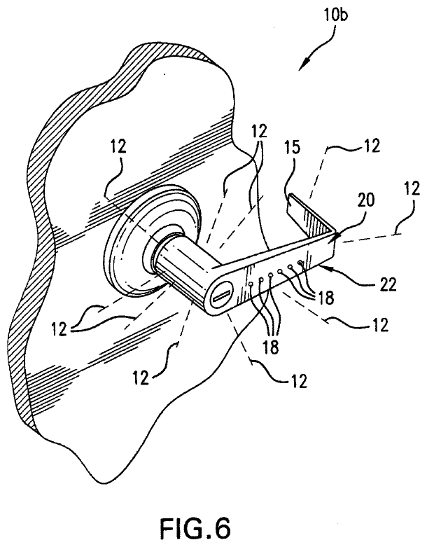

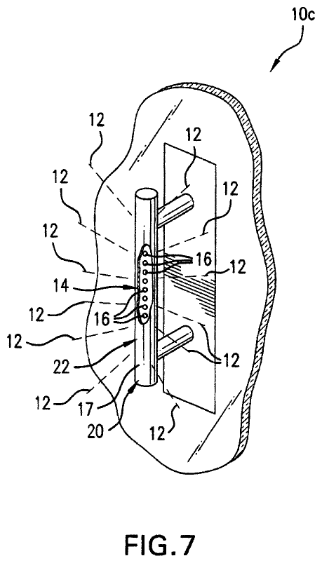

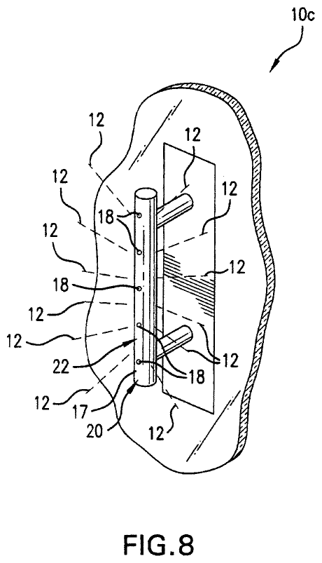

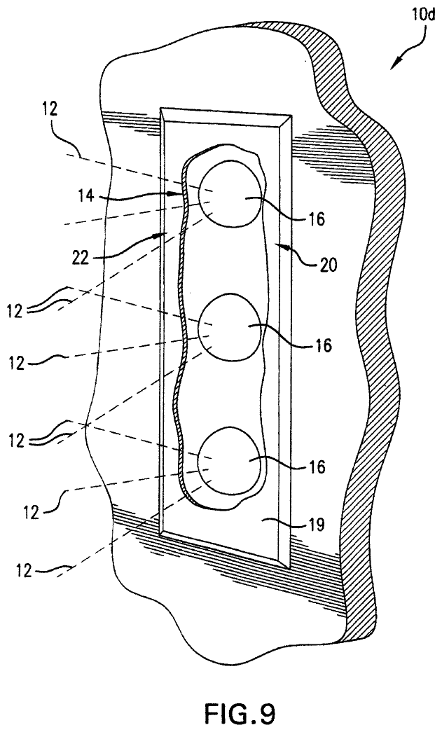

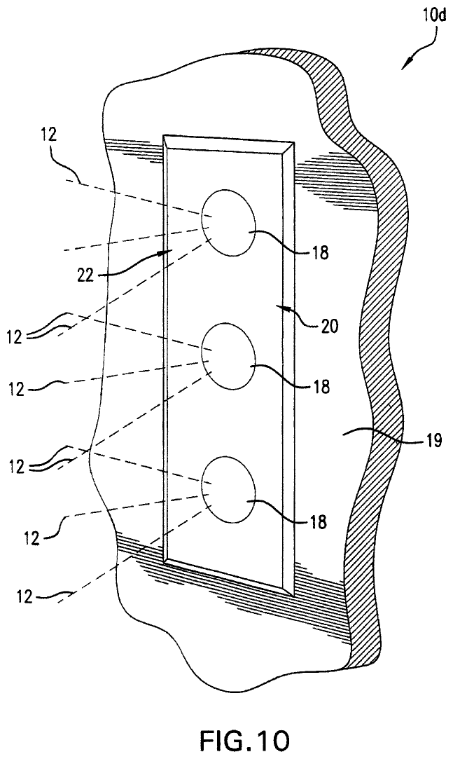

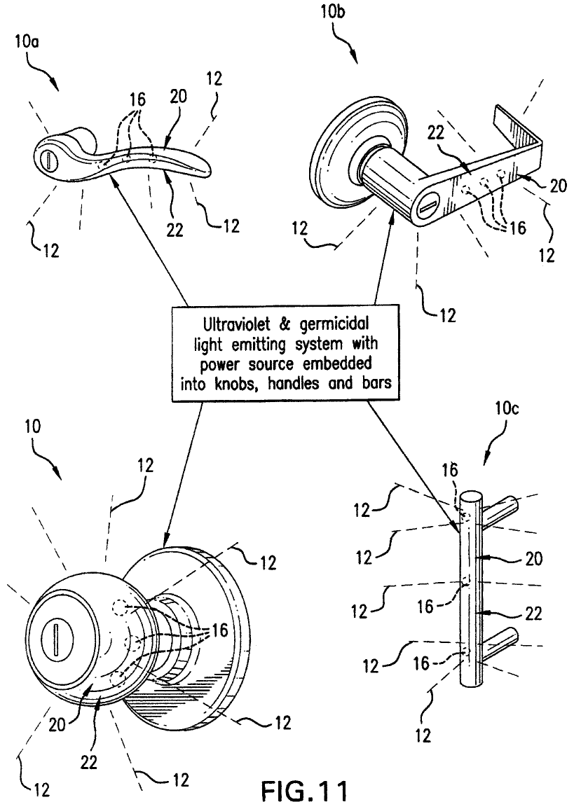

FIG. 2 is a further preferred embodiment doorknob 11 without showing cutaway 14 in ultraviolet C light permeable material 22. FIG. 3 illustrates the present invention adapted to and incorporated into a contoured door lever 13, while FIG. 4 shows another preferred embodiment of the present invention adapted to, and integrated, into a contoured door lever 13. FIG. 5 is a perspective view of the present invention adapted to, and incorporated into, a rectangular door lever 15. FIG. 6 shows another perspective view of the present invention adapted to, and integrated, into a rectangular door lever 15, while FIG. 7 illustrates the present invention as adapted to, and incorporated into, a door bar handle 17. FIG. 8 shows another perspective view of the present invention adapted to, and integrated into, a door bar handle 17. FIG. 9 illustrates the present invention as adapted to, and incorporated into, a door push pad plate 19. FIG. 10 shows another view of the present invention adapted to, and integrated into, a door push pad plate 19. Finally, FIG. 11 summarizes various configurations wherein the instant invention is utilized.

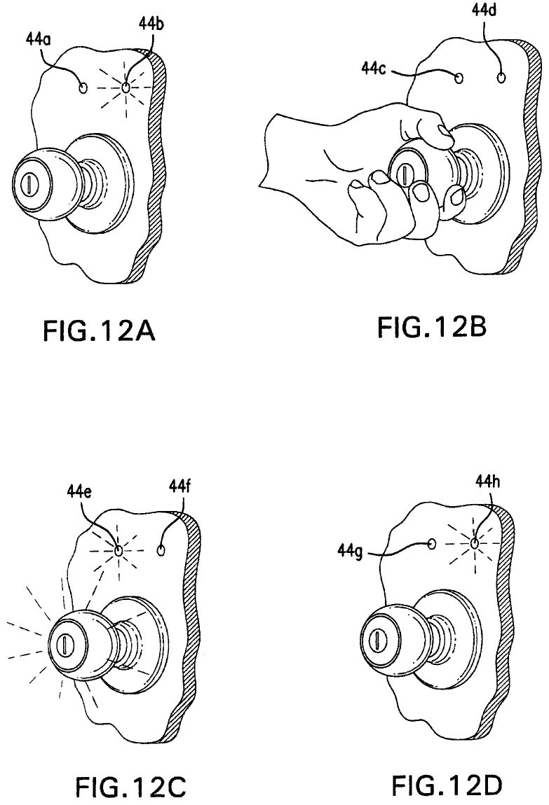

FIG. 12A indicates the condition of a doorknob, namely, when doorknob red light 44a is off and green light 44b is illuminated doorknob is germ free and ready to be accessed. FIG. 12B indicates an un-sanitized doorknob transferring pathogens to the user's hand and neither light 44c nor 44d is illuminated. FIG. 12C indicates the cleanliness condition of the doorknob. Light 44e is shown as being illuminated indicating source 12 being energized for, by way of example, 10 to 20 seconds which is sufficient time to destroy pathogens during said 10 to 20 seconds and at same time light 44f is not illuminated. FIG. 12D is still another view as to cleanliness condition of the doorknob with light 44g being “off” and light 44h is illuminated indicating that surface is germ-free and may safely be touched.

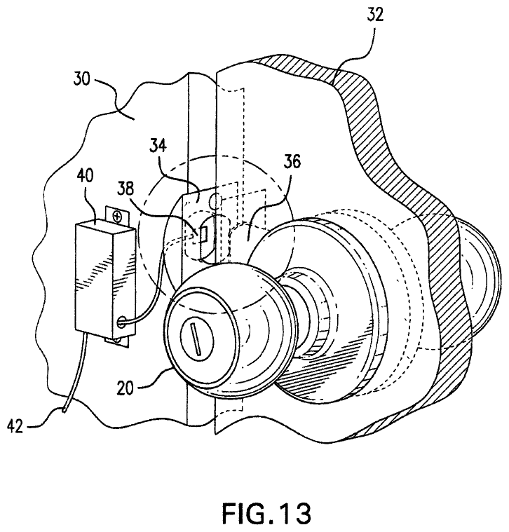

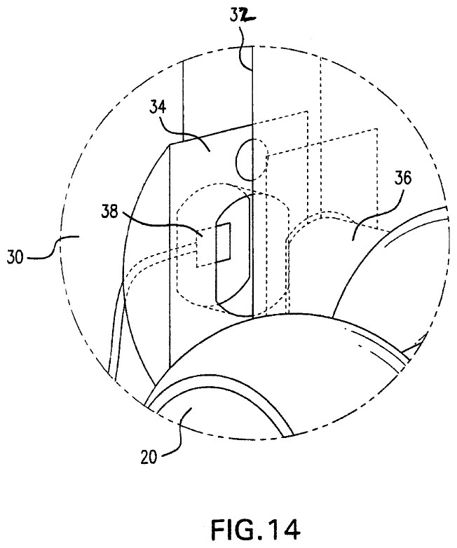

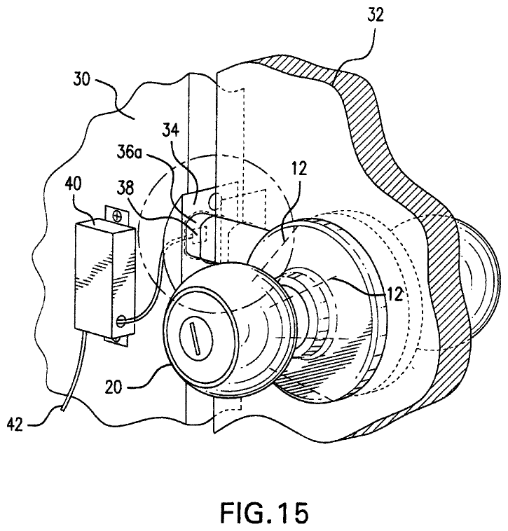

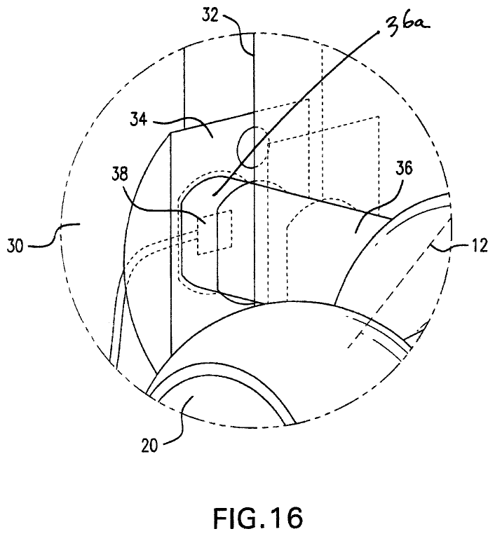

FIG. 13 illustrates a method of transmitting of power 42 (generally, 120 volts AC but not limited thereto) by means of AC to DC converter 40 from door frame 30 to door 32. Striker plate 34 surrounds electrical switch 38 and when activated permits electricity to flow into lock barrel 36 and then to door 32 and energizing ultraviolet C germicidal light source 18. In the position as shown switch 38 is not activated and power is not in communication with door 32. FIG. 14 indicates non-engaged lock barrel 36 displaced from electrical switch 38 and accordingly electric current is not flowing to door 32. FIG. 15 indicates engaged lock barrel 36a in contact with switch 38 facilitating electric current to flow to door 32 and then to the source of the ultraviolet C germicidal light 12. Finally, FIG. 16 illustrates the positions of un-engaged lock barrel 36 and engaged lock barrel 36a, which provides and transmits electric power to ultraviolet C disinfecting light source 16 to ultraviolet C germicidal light 12.

While only several embodiments of the present invention have been shown and described, it will be obvious to those skilled in the art that many modifications may be made to the present invention without departing from the spirit and scope thereof.

1. A self-contained ultraviolet C light emitting system for door access, comprising:
a door for entering and exiting a premise;
a door access device associated with the door wherein a user is able to come into physical contact with the door access device;
a source of sanitizing ultraviolet C emitting light associated with the door access device positioned so that the door access device is sanitized and germ free; and,
means for electrically energizing the source of the sanitizing ultraviolet light includes a switch that electrically powers the door access device, the switch comprising a lock barrel that comes into contact with, and actuates, the switch permitting electric current to energize the source of sanitizing ultraviolet C light, the lock barrel being in an engaged position with the switch for facilitating electric current to flow to the door, while in a non-engaged position the lock barrel is displaced from the switch thereby preventing electric current from flowing to the door.

2. The self-contained ultraviolet C light emitting system according to claim 1, wherein the source of sanitizing ultraviolet C emitting light is selected from the group consisting of light emitting diodes, fiber optics, glass fiber and mercury bulbs.

3. The self-contained ultraviolet C light emitting system according to claim 1, wherein the door access device is selected from the group consisting of doorknobs, contoured door levers, rectangular door levers, door bar handles and door push plates.

4. The self-contained ultraviolet C light emitting system according to claim 1, wherein the door access device is made of an ultraviolet C light permeable material so that the ultraviolet C light emitted from the source of the ultraviolet C light comes into contact and sanitizes the door access device.

5. The self-contained ultraviolet C light emitting system according to claim 1, wherein the door access device has incorporated into the door accessory device the source of the ultraviolet C sanitizing light.

6. (canceled)

7. The self-contained ultraviolet C light emitting system according to claim 1, further comprising means to visually determine the electrical condition of the ultraviolet C light emitting system.
